(19) United States
(12) Patent Application Publication
(10) Pub. No.: US 2023/0401853 A1
(43) Pub. Date: Dec. 14, 2023

(54) SYSTEMS AND METHODS FOR MONITORING FACE MASK WEARING

(71) Applicant: SIGNIFY HOLDING B.V., EINDHOVEN (NL)

(72) Inventors: DAKSHA YADAV, BOSTON, MA (US); JIN YU, LEXINGTON, MA (US); ABHISHEK MURTHY, ARLINGTON, MA (US); PETER DEIXLER, ARLINGTON, MA (US)

(21) Appl. No.: 18/032,057

(22) PCT Filed: Oct. 15, 2021
(86) PCT No.: PCT/EP2021/078568

§ 371 Date: Apr. 14, 2023

Related U.S. Application Data
(60) Provisional application No. 63/093,811, filed on Oct. 20, 2020.

(30) Foreign Application Priority Data
Oct. 26, 2020 (EP) 20203871.7

(51) Int. Cl. G06V 20/52; G06V 40/16; G06V 10/762; G06V 10/10; G06V 10/26; G06V 10/764; G06V 10/147; A61L 2/24; A61L 2/08; A61L 2/14

(52) U.S. Cl. CPC G06V 20/52; G06V 40/171; G06V 10/762; G06V 10/16; G06V 10/26; G06V 10/764; G06V 10/147; A61L 2/24; A61L 2/08; A61L 2/14; G10L 25/51

(57) ABSTRACT

A system for monitoring face mask wearing of a monitored person is provided. The system includes a controller communicatively coupled to one or more multipixel thermopile sensors (“MPTs”). The controller is configured to (1) detect a monitored person region within a heat map based on one or more data sets captured by the MPTs; (2) locate a head region within the monitored person region by identifying a high intensity pixel cluster within the monitored person region; (3) determine a facing-direction of the head region based on a major axis of the monitored person region or a minor axis of the monitored person region; (4) locate a mouth region within the head region based on the facing-direction; (5) determine an exhalation region of the heat map based on the mouth region; (6) determine a mask state of the monitored person based on the exhalation region and a temperature gradient classification model.

### FIELD OF THE DISCLOSURE

The present disclosure is directed generally to monitoring face mask wearing using advanced sensor bundles embedded in a lighting Internet of Things system.

### BACKGROUND

During the recent COVID-19 pandemic, studies have shown that a substantial percentage of people with COVID-19 lack symptoms. Additionally, studies have also shown that those who eventually develop symptoms can spread the virus to others even before exhibiting symptoms. Due to this, the Center for Disease Control (CDC) in the United States recommends everyone, sick or healthy, to wear face coverings or masks in public spaces (such as grocery stores and offices), along with exercising other social distancing measures. The aforementioned face mask is intended to cover the mouth and nose, and can block the release of virus-filled droplets into the air when the wearer coughs, sneezes, or even talks. This blocking can help in slowing the spread of COVID-19 and other communicable diseases.

Wearing a face mask changes the airflow and breathing resistance of an person's exhalations. The change varies widely depending on structural features of the face mask, such as respiratory valves, overall shape, and/or materials used. The face masks with a respiratory valve typically results in a lower change rate of breathing resistance. Moreover, a cup type mask typically results in a lower change rate of breathing resistance than a folding mask. Furthermore, a cotton mask results in a lower change rate of breathing resistance than a nonwoven fabric mask. However, in all cases when a mask is worn, the airflow is significantly lowered compared to the non-mask baseline.

As the usage of face masks becomes more critical and prevalent, there is a need to automatically monitor people for face mask wearing. The existing monitoring systems use a thermal camera frontally looking at faces or an RGB camera to detect masks at the entrance of a room. In these technologies, the camera is required to look at the face of the monitored people. However, for privacy of the person and ease of positioning monitoring equipment, it would be advantageous to discreetly monitor the people from an overhead position, rather than requiring a clear view of their face.

### SUMMARY OF THE DISCLOSURE

The present disclosure is directed generally to monitoring face mask wearing using advanced sensor bundles (“ASBs”) embedded in a lighting Internet of Things (“IoT”) system. The ASBs include one or more multipixel thermopile sensors (“MPTs”). The system generates a heat map for an area with a monitored person based on data sets captured by the MPTs. The system then detects the monitored person region within the heat map. The system then locates a head region of the monitored person, and determines the facing-direction of the head region. Based on the facing-direction, the system then locates a mouth region within the head region. The system then determines an exhalation region of the heat map based on the mouth region. The exhalation region represents the portion of the heat map impacted by the breath of the monitored person. The system then determines a mask state (masked, partially-masked, unmasked, or improperly masked) of the monitored person based on the exhalation region and a temperature gradient classification model. If the monitored person is determined to be partially-masked, unmasked, or improperly masked the system may activate one or more enforcement and/or disinfectant measures, such as transmitting a warning signal or configuring one or more light sources or ionizers to operate in a disinfecting mode. In further examples, the ASBs may also include one or more microphones configured to capture audio signals related to the speech or breath of the monitored people. The system may use the audio signals to augment or confirm the mask state determination.

Generally, in one aspect, a system for monitoring face mask wearing of a monitored person is provided. The system may include a controller. The controller may be communicatively coupled to one or more MPTs. The MPTs may be arranged in one or more luminaires. The luminaires may be positioned above the monitored person.

The controller may be configured to detect a monitored person region within a heat map. The heat map may be based on one or more data sets captured by the one or more MPTs.

The controller may be further configured to locate a head region within the monitored person region. According to an example, the head region may be located by identifying a high intensity pixel cluster within the monitored person region.

The controller may be further configured to determine a facing-direction of the head region. According to an example, the facing-direction of the head region may be determined based on a major axis of the monitored person region or a minor axis of the monitored person region.

The controller may be further configured to locate a mouth region within the head region based on the facing-direction.

The controller may be further configured to determine an exhalation region of the heat map based on the mouth region.

The controller may be further configured to determine a mask state of the monitored person. The mask state may be determined based on the exhalation region and a temperature gradient classification model. According to an example, the temperature gradient classification model may be an artificial neural network. According to an example, the temperature gradient classification model may be a support vector machine.

According to an example, detecting the monitored person region may include image-stitching the one or more data sets to generate the heat map.

Detecting the monitored person region may further include clustering one or more pixels of the heat map into one or more object clusters. The one or more pixels may be clustered based on an intensity of the pixels.

Detecting the monitored person region may further include segmenting one or more object boundaries. The object boundaries may be segmented based on the one or more object clusters.

Detecting the monitored person region may further include classifying the pixels within one of the object boundaries as the monitored person region. The pixels may be classified based on a person classification model. According to an example, the person classification model may be a Light Gradient Boosting Machine (LGBM).

According to an example, the exhalation region may be further located based on an audio arrival angle of one or more speech audio signals. The speech audio signals may be captured by one or more microphones. The microphones may be communicatively coupled to the controller. The speech audio signals may correspond to speech of the monitored person.

According to an example, the controller may be further configured to transmit a warning signal. The warning signal may be transmitted based on the mask state of the monitored person.

According to an example, one or more light sources and/or one or more ionizers communicatively coupled to the controller may operate in a disinfecting mode. The light sources and/or ionizers may operate in a disinfecting mode based the mask state.

According to an example, the determination of the mask state of the monitored person may be further based on one or more breath audio signals captured by one or more microphones and a breathing audio classification model. The breath audio signals may correspond to breathing of the monitored person. The microphones may be communicatively coupled to the controller.

Generally, in another aspect, a method for monitoring face mask wearing of a monitored person is provided. The method may include detecting, via a controller communicatively coupled to one or more MPTs, a monitored person region within a heat map, wherein the heat map is based on one or more data sets captured by the one or more MPTs. The method may further include locating, via the controller, a head region within the monitored person region. The method may further include determining, via the controller, a facing-direction of the head region. The method may further include locating, via the controller, a mouth region within the head region based on the facing-direction. The method may further include determining, via the controller, an exhalation region of the heat map based on the mouth region. The method may further include determining, via the controller, a mask state of the monitored person based on the exhalation region and a temperature gradient classification model.

According to an example, detecting the monitored person region may include image-stitching the one or more data sets to generate the heat map. Detecting the monitored person region may further include clustering one or more pixels of the heat map into one or more object clusters based on an intensity of the pixels. Detecting the monitored person region may further include segmenting one or more object boundaries based on the one or more object clusters. Detecting the monitored person region may further include classifying, based on a person classification model, the pixels within one of the object boundaries as the monitored person region.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a top-level schematic of a system for monitoring and enforcing face mask wearing, in accordance with an example.

FIG. 2 is a schematic of a luminaire in a system for monitoring and enforcing face mask wearing, in accordance with an example.

FIG. 3 is a schematic of a controller in a system for monitoring and enforcing face mask wearing, in accordance with an example.

FIG. 4 is an illustration of a system for monitoring and enforcing face mask wearing, in accordance with an example.

FIG. 5 is an illustration of the impact of mask wearing on a person's exhalation, in accordance with an example.

FIG. 6 is a heat map generated by a system for monitoring and enforcing face mask wearing, in accordance with an example.

FIG. 7 is a flowchart of a method for monitoring and enforcing face mask wearing, in accordance with an example.

FIG. 8 is a flowchart for the detecting of a monitored person region aspect of the method for monitoring and enforcing face mask wearing, in accordance with an example.

### DETAILED DESCRIPTION OF EMBODIMENTS

The present disclosure is directed generally to monitoring face mask wearing using advanced sensor bundles (“ASBs”) embedded in a lighting Internet of Things (“IoT”) system. The ASBs include one or more multipixel thermopile sensors (“MPTs”). The system generates a heat map for an area with a monitored person based on data sets captured by the MPTs. The system then detects the monitored person region within the heat map. The monitored person region may be detected by (1) image-stitching the data sets to generate the heat map; (2) clustering pixels of the heat map into object clusters based on an intensity of the pixels; (3) segmenting object boundaries based on the one or more object clusters; and (4) classifying the pixels within one of the object boundaries as the monitored person region. The system then locates a head region of the monitored person by identifying a high intensity pixel cluster within the monitored person region. Based on the facing-direction, the system then locates a mouth region within the head region. The system then determines an exhalation region of the heat map based on the mouth region. The exhalation region represents the portion of the heat map impacted by the breath of the monitored person. The system then determines a mask state (masked, partially-masked, unmasked, or improperly masked) of the monitored person based on the exhalation region and a temperature gradient classification model. If the monitored person is determined to be unmasked, partially-masked, or improperly masked, the system may activate one or more enforcement and/or disinfectant measures, such as transmitting a warning signal or configuring one or more light sources or ionizers to operate in a disinfecting mode. In further examples, the ASBs may also include one or more microphones configured to capture audio signals related to the speech or breath of the monitored people. The system may use the audio signals to augment the mask state determination.

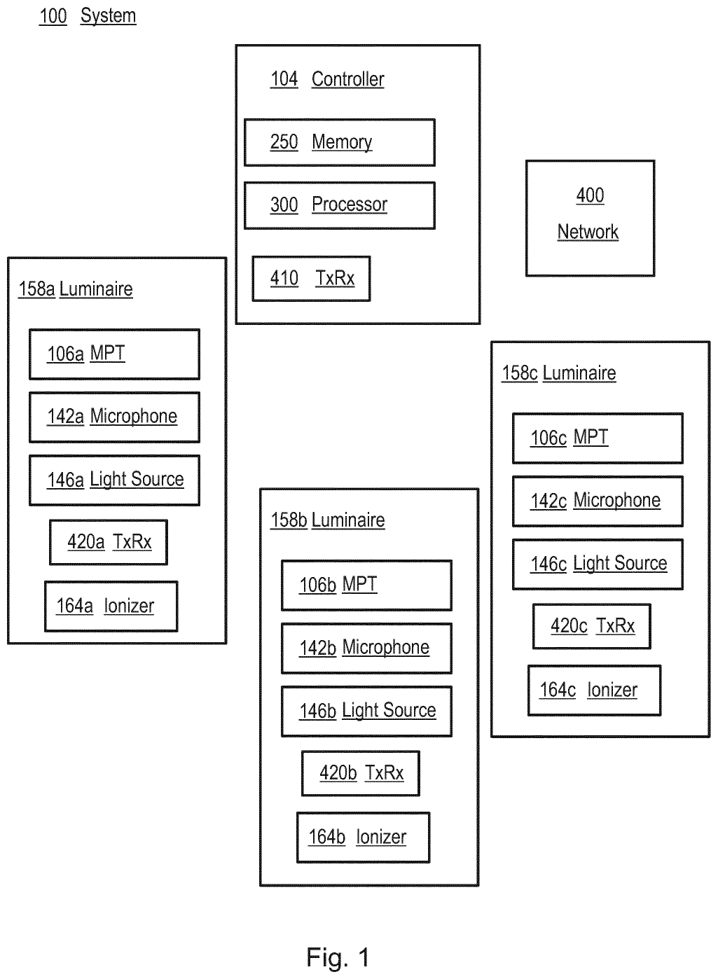

Generally, in one aspect, and with reference to FIGS. 1-4, a system 100 for monitoring face mask wearing of a monitored person 102 is provided. Broadly, the system 100 may include a controller 104 and one or more luminaires 158. Each of the luminaires 158 may include components such as MPTs 106, microphones 142, light sources 146, and/or ionizers 164. The controller 104 may be capable of communication with the components of the luminaires 158 via wired or wireless network 400. FIG. 1 depicts an example system 100 which includes three luminaires 158a-c, each luminaire 158a-c having an MPT 106a-c, microphone 142a-c, light source 146a-c, ionizer 164a-c, and transceiver 420a-c. Within each luminaire 158, the MPT 106 and microphone 142 may be packaged together as an ASB.

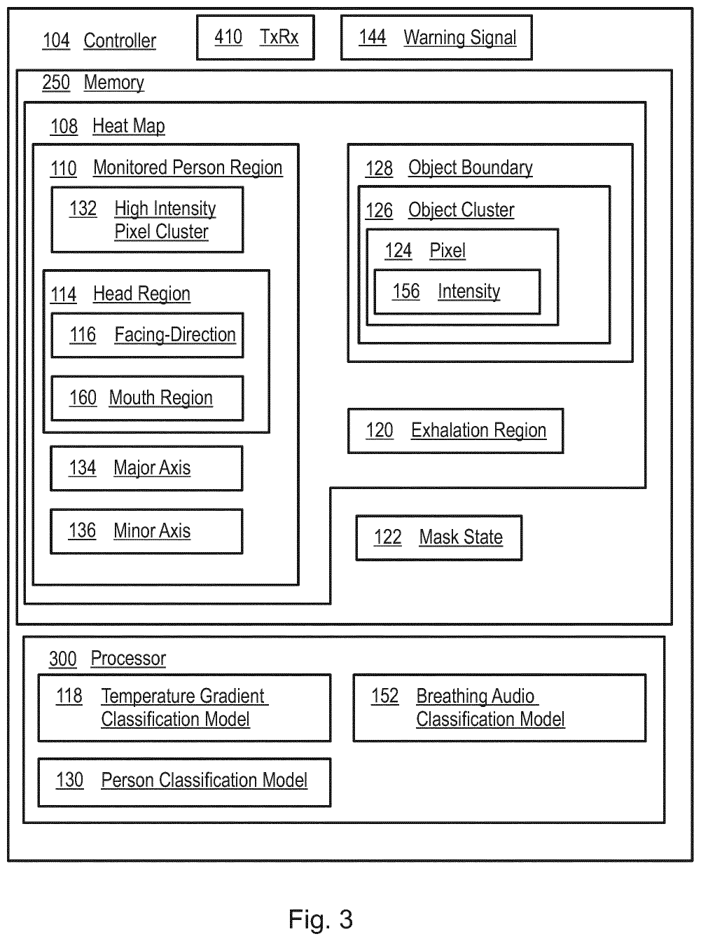

With reference to FIGS. 1 and 3, the controller 104 may include a memory 250, a processor 300, and a transceiver 410. The memory 250 and processor 300 may be communicatively coupled via a bus to facilitate processing of data stored in memory 300. Transceiver 410 may be used to receive data from the one or more MPTs 106 or microphones 142 via the network 400. The data received by the transceiver 410 may be stored in memory 250 and/or processed by processor 300. In an example, the transceiver 410 may facilitate a wireless connection between the controller 106 and the network 400.

The network 400 may be configured to facilitate communication between the controller 104, the one or more MPTs 106, the one or more microphones 142, the one or more light sources 146, and/or any combination thereof. The network 400 may be a wired and/or wireless network following communication protocols such as cellular network (5G, LTE, etc.), Bluetooth, Wi-Fi, Zigbee, and/or other appropriate communication protocols. In an example, the MPT 106 may wirelessly transmit, via the network 400, a data set 112 to the controller 106 for storage in memory 250 and/or processing by the processor 300.

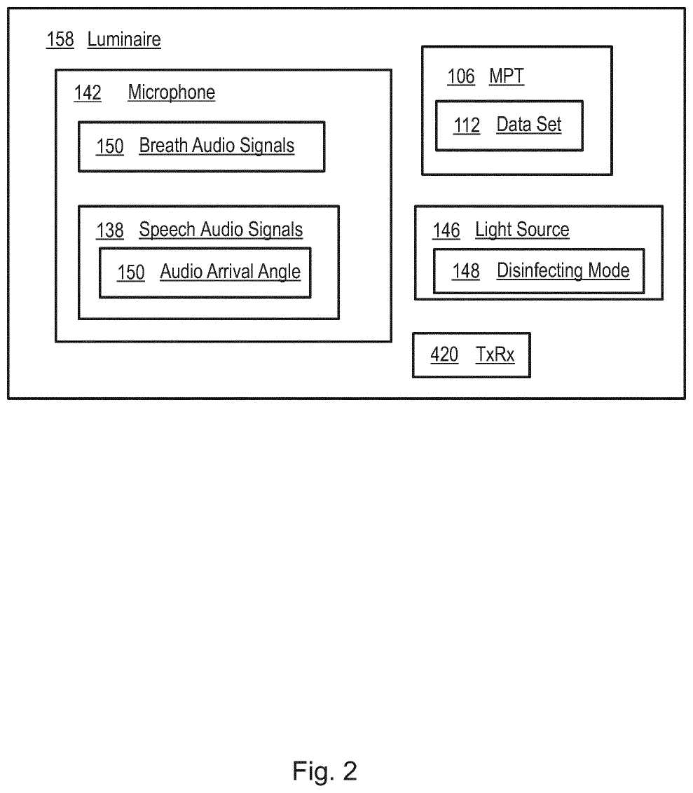

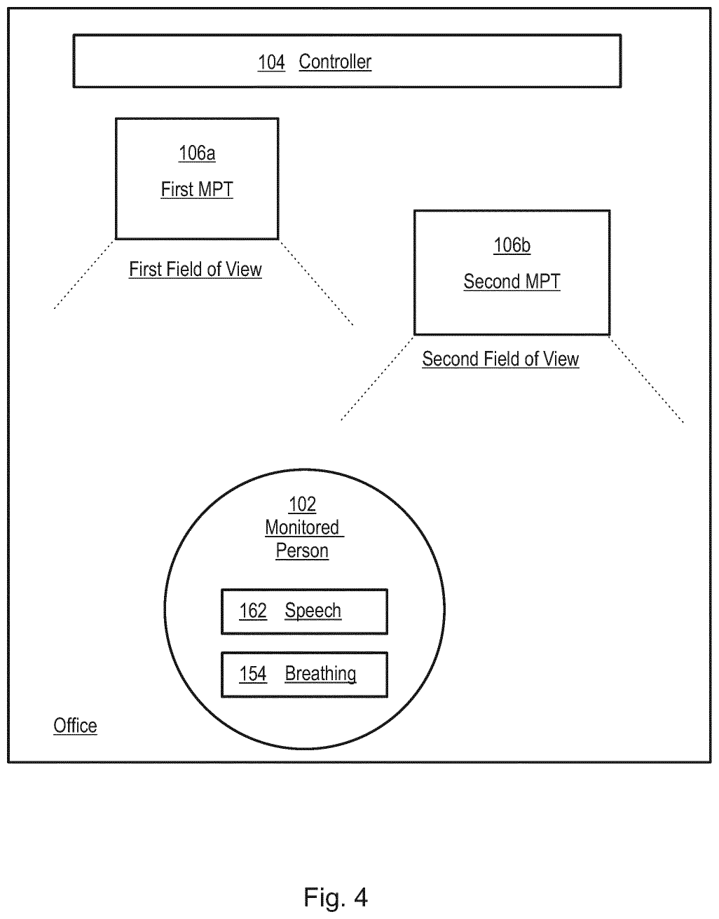

The controller 104 may be communicatively coupled to one or more MPTs 106 via network 400. As shown in FIG. 2, the MPTs 106 may be arranged in one or more luminaires 158. As shown in FIG. 4, the luminaires 158 may be positioned above the monitored person 102. In one example, the luminaires 158 may be hanging from the ceiling of an office. In another example, the luminaires 158 may be mounted on the upper regions of the walls of the office. The MPTs may be low resolution, such as 24×48 pixels.

The controller 106 may be configured to detect a monitored person region 110 within a heat map 108. The monitored person region 110 represents the position of the monitored person 102 in a monitored area, such as an office.

The heat map 108 includes a plurality of pixels 124 and may be based on one or more data sets 112 captured by the one or more MPTs 106. The heat map 108 may be two-dimensional or three-dimensional depending on the captured data sets 112.

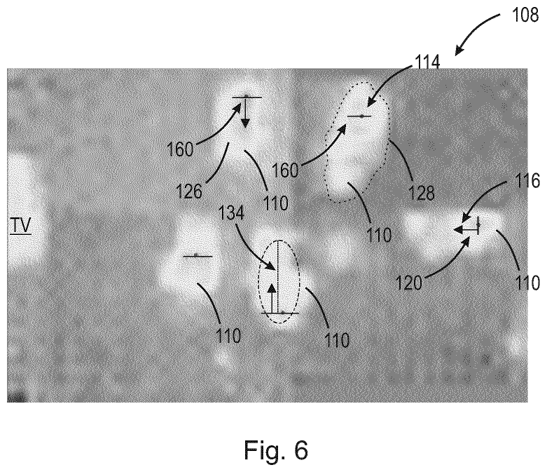

An example heat map 108 is shown in FIG. 6. The example heat map 108 contains five example monitored person regions 110 representing five monitored persons 102 in a meeting room seated around a table. The rectangular region on the far left of the heat map 108 represents a television screen. The data sets 112 captured by each MPT 106 correspond to its field of view. For example, as shown in FIG. 4, first MPT 106a has a first field of view, while second MPT 106b has a second field of view. The difference in these fields of view results in their corresponding data sets 112 containing data representing different aspects of the monitored area. In a preferred example, the system 100 includes a high number of MPTs (tens or even hundreds) with overlapping fields of view positioned at ceiling level. In this preferred example, the fields of view may overlap by 15 percent. According to an example, detecting the monitored person region 110 may include image-stitching the one or more data sets 112 to generate the heat map 108. By using image-stitching, the processor 300 combines the data sets 112 corresponding to many different fields of view into a single, coherent heat map 108. The resulting heat map 108 thus represents a much larger area than a heat map 108 based on a data set 112 captured by a single MPT 106.

Detecting the monitored person region 110 may further include clustering one or more pixels 124 of the heat map 108 into one or more object clusters 126. An example object cluster 126 is shown in FIG. 6. The one or more pixels 124 may be clustered based on an intensity 156 of the pixels 124. In an example, proximate or adjacent pixels 124 with an intensity of above a certain threshold relative to the heat map 108 may be clustered together to form object clusters 126 representative of people. As shown in FIG. 6, the lighter (higher intensity) pixels 124 are presumed to represent people within the monitored environment, and are therefore clustered together.

Detecting the monitored person region 110 may further include segmenting one or more object boundaries 128. The object boundaries 128 may be segmented based on the one or more object clusters 126. An example object boundary 128 is shown in FIG. 6. This example object boundary 128 surrounds pixels 124 of similar intensity 156 forming an object cluster 126.

Detecting the monitored person region 110 may further include classifying the pixels 124 within one of the object boundaries 128 as the monitored person region 110. The pixels 124 may be classified based on a person classification model 130. The person classification model 130 may be a machine learning algorithm. According to an example, the person classification model 130 may be a Light Gradient Boosting Machine (LGBM). The person classification model 130 may be any other machine learning classification algorithm configured to identify a pixel 124 as corresponding to a person based on intensity 156.

The person classification model 130 may analyze the pixels 124 based on a number of factors, including intensity 156, size of the object cluster 126, and shape of the object cluster 126. For example, if a few pixels 124 with person-like intensity 156 are part of a very small pixel cluster 126, the person classification model 130 should classify those pixels 124 as NOT part of the monitored person region 110. Rather than representing a person, these pixels 124 may instead represent a small light source, such as a desk lamp or a computer monitor.

The controller 106 may be further configured to locate a head region 114 within the detected monitored person region 110. According to an example, the head region 114 may be located by identifying a high intensity pixel cluster 132 within the monitored person region 110, as the head is typically one of the warmest parts of the human body. Each monitored person region 110 of FIG. 6 includes a dot designating the peak temperature points of the head regions 114.

The controller 106 may be further configured to determine a facing-direction 116 of the head region 114. The facing-directions 116 of the head regions 114 in FIG. 6 are represented by the arrows within monitored person regions 110.

Determining the facing-direction 116 of the head region may involve determining whether the monitored person 102 is standing or sitting. The system 100 may determine whether the monitored person 102 is standing or sitting based on a number of factors, including the size and shape of the monitored person region 110, as well as the location of the head region 114. For example, a larger, rectangular-shaped monitored person region 110 may be indicative of a monitored person 102 sitting, as the rectangular shape may be due to a person's legs extending beyond their upper body. Conversely, a smaller, squarish or circular monitored person region 110 may be indicative of a monitored person 102 standing upright. For example, the monitored persons 102 of FIG. 6 are all sitting around a table, thus leading to the rectangular shapes of their corresponding monitored person regions.

According to a further example, the facing-direction 116 of the head region 114 may be determined based on a major axis 134 of the monitored portion region 110 or a minor axis 136 of the monitored person region 110. The major axis 134 and minor axis 136 may be determined by (1) inscribing an ellipse approximately around monitored person region 110 and (2) drawing the major and minor axes of the ellipse. If the monitored person 102 is determined to be standing, the direction the major axis 134 originating from the head region may indicate the facing-direction 116 of the head region 114. Similarly, if the monitored person 102 is determined to be standing, the direction the minor axis 136 originating from the head region may indicate the facing-direction 116 of the head region 114.

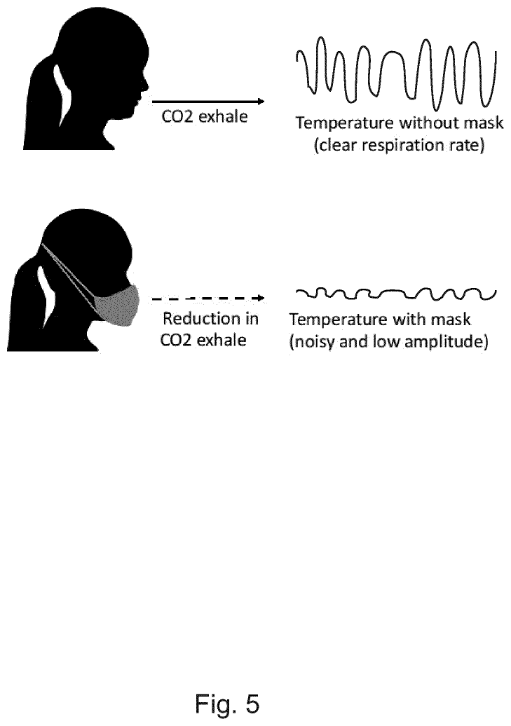

The controller 104 may be further configured to locate a mouth region 160 within the head region 114 based on the facing-direction 116. The controller 106 may be further configured to determine an exhalation region 120 of the heat map 108 based on the mouth region 160. FIG. 5 demonstrates the impact of a mask on the exhaled breath of a person. For example, without a mask, the temperature of the exhalation region 120 will increase to a much higher degree than when the user is masked. If the user is partially-masked (for instance, if their nose is exposed), the temperature of the exhalation region 120 will be higher than if the user was masked, but not to the degree as if they were fully unmasked.

The exhalation region 120 is the portion of the heat map 108 which will experience a change in intensity when an unmasked person exhales. The exhalation region may be determined by locating the mouth region 160 of the monitored person region 110 based on the facing-direction 116 of the monitored person region 110. For example, the mouth region 160 may be a few (less than 10) pixels within the head region 114. Further, the mouth region 160 will correspond to the facing-direction 116 of the monitored person region 110. As shown in FIG. 6, the mouth region 160 may be found in the facing-direction of a monitored person region 110, just below the peak temperature points of the head region 114. The exhalation region 120 accordingly includes the neighboring pixels of the located mouth region 160. Each monitored person region 110 of FIG. 6 includes an exhalation region 120 corresponding to a mouth region 160.

The controller 106 may be further configured to determine a mask state 122 of the monitored person 102 as “masked”, “partially-masked”, “unmasked”, or “improperly masked”. In this context, a mask state 122 of “masked” means that the monitored person 102 is wearing a face mask suitable for protecting others from viral infections according to governmental health guidelines, such as a surgical mask of appropriate thickness. A mask state 122 of “unmasked” means that the monitored person 102 is not wearing a face mask of any kind. A mask state 122 of “partially-masked” means that the monitored person 102 is wearing a face mask, but the mask is positioned incorrectly, leaving their mouth and/or nose at least partially exposed.

A mask state 122 of “improperly masked” means that the monitored person 102 is wearing a face mask that does not adequately protect others from viral infections. For example, some N95 mask or respirator models include exhalation valves to make breathing out easier and reduce heat build-up. While such mask models protect the wearer, they protect other people to a lesser degree than an N95 mask or respirator without exhalation valves. Hence, it is desirable to use the disclosed system 100 to detect whether a mask with an exhalation valve is used, and thus assign the mask state 122 to “improperly masked”. The heat exhaust level from such a mask with valves is typically in between that of a mask-less person and a masked person.

The controller 106 may be configured to evaluate the monitored person for additional mask states 122 as appropriate.

The mask state may be determined based on the exhalation region 120 and temperature gradient classification model 118. According to an example, the temperature gradient classification model 118 may be an artificial neural network. According to an example, the temperature gradient classification model 118 may be a support vector machine. The temperature gradient classification model 118 may be any other algorithm (such as a machine learning algorithm) configured to differentiate between masked, partially-masked, unmasked, and improperly masked states based on the temperature gradient of the pixels 124 of the exhalation region 120.

If, based on the temperature gradients of the pixels 124 of the exhalation region 120, the temperature gradient classification model 118 determines that the monitored person 102 is wearing a mask incorrectly (such as leaving their nose uncovered), the system 100 may determine the mask state 122 to be partially-masked. Similarly, if the temperature gradient classification model 118 determines that the monitored person 102 is wearing a mask with exhalation valves, the system 100 may determine the mask state to be improperly masked.

In a preferred example, the captured data sets 112 are utilized to generate a series of heat maps 108 over a time period. The temperature gradient classification model 118 may then be used to analyze the change in intensity 156 of the pixels 124 of the exhalation region 120 over the time period to more accurately determine the mask state 122 of the monitored person 102. For example, the temperature gradient classification model 118 may be used to determine if change of the intensity 156 of the pixels 124 of the exhalation region 120 shows a clear human respiration rate of 12 to 20 breaths per minute. If so, the mask state 122 may be determined to be unmasked, partially-masked, or improperly masked, depending on the amplitude, frequency, and/or shape of the change of intensity 156. If not, the mask state 122 may be determined to be masked. In this way, the system 100 may determine the mask state 122 of a monitored person 102 wearing a surgical mask to be “masked”, while also determining the mask state 122 of a monitored person wearing an N95 mask with exhaust valves to be “improperly masked”.

For example, when the monitored person 102 breathes out, the impacted portion of the exhalation region 120 is largest right after the exhale has finished. Similarly, the impacted portion of the exhalation region 120 is smallest during the start of the exhale. Hence, the dynamic changes in size of the impacted portion of the exhalation region 120 indicate whether a mask is worn. Therefore, in a preferred example, the temperature gradient classification model 118 may analyze and determine how the impacted portion of the exhalation region 120 expands and contracts over time.

The exhalation region 120 may be refined or adapted based on the stability of the head region 114 over time by tracking the location of the mouth region 160 of the monitored person region 110.

According to an example, the exhalation region 118 may be further determined or confirmed based on an audio arrival angle 140 of one or more speech audio signals 138. The speech audio signals 138 may correspond to speech 162 of the monitored person 102. The speech audio signals 138 may be captured by one or more microphones 142. In a preferred example, the microphones 142 must be capable of estimating audio arrival angle 140 with a one degree margin of error. As shown in FIGS. 1 and 2, the microphones 142 may be arranged in the luminaires 158, and may be communicatively coupled to the controller 104. Analyzing the audio arrival angle 140 allows the system 100 to more accurately locate the mouth portion 160 of the head region 114, which, as the monitored person 102 both speaks and exhales from their mouth, corresponds to the exhalation region 120. The audio arrival angle 140 of the speech audio signals 138 may be determined through any number of known signal processing means.

According to an example, the controller 104 may be further configured to transmit a warning signal 144. The warning signal 144 may be transmitted if the mask state 122 of the monitored person 102 is partially-masked, unmasked, or improperly masked. The warning signal 144 may be a wireless signal transmitted by transceiver 410. The warning signal 144 may be received by a central monitoring station, which may be configured to alert a supervisor, co-worker, or co-occupant that the monitored person is partially-masked, unmasked, or improperly masked. Further, the warning signal 144 may be received by a device operated by the monitored person 102, causing the device to generate audio and/or visual feedback notifying the person that they are partially-masked, unmasked, or improperly masked.

According to an example, one or more light sources 146 and/or one or more ionizers communicatively coupled to the controller 104 may operate in a disinfecting mode 148 if the mask state 122 of the monitored person 102 is partially-masked, unmasked, or improperly masked. In this example, the controller 104, via transceiver 410, may wirelessly transmit a signal to luminaire 158, containing light source 146. The command may be wirelessly received by the luminaire 158, via transceiver 420, and cause the light source 146 to emit disinfecting ultraviolet light. The signal may also trigger the light source 146 to blink or change colors, thus informing any nearby people that a person in the vicinity is partially-masked, unmasked, or improperly masked. The signal may also trigger the ionizer 164 to disinfect the air surrounding the luminaire 158.

According to an example, the determination of the mask state 122 of the monitored person 102 may be further based on one or more breath audio signals 150 captured by one or more microphones 142 and a breathing audio classification model 152. The breath audio signals 150 may correspond to breathing 154 of the monitored person 102. The microphones 142 may be communicatively coupled to the controller 104. In this case, the system 100 analyzes the audio characteristics, such as volume or frequency, of the breathing 154 to aid in the determination of the mask state 122. As shown in FIG. 5, the breathing 154 of an unmasked or partially-masked person will be significantly louder with a more easily detectable frequency as compared to a masked person.

In the aforementioned example, an array of microphones 142 may detect an audio arrival angle corresponding to the breath audio signals 150. The microphones 142 may then perform beamforming to focus on the sound from people's mouths whenever the monitored person 102 is not talking. A clear breathing 154 sound will be clearly heard without a mask. Accordingly, simple binary classification for breathing audio signals 150 can also help determine the mask state 122.

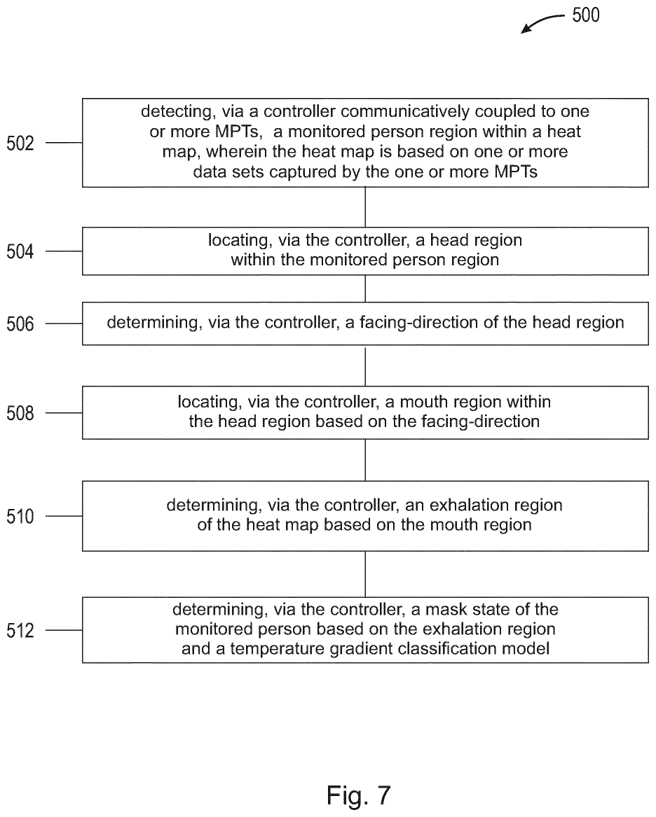

Generally, in another aspect, and with reference to FIG. 7, a method 500 for monitoring face mask wearing of a monitored person is provided. The method 500 may include detecting 502, via a controller communicatively coupled to one or more MPTs, a monitored person region within a heat map, wherein the heat map is based on one or more data sets captured by the one or more MPTs. The method 500 may further include locating 504, via the controller, a head region within the monitored person region. The method 500 may further include determining 506, via the controller, a facing-direction of the head region. The method 500 may further include locating 508, via the controller, a mouth region within the head region based on the facing-direction. The method 500 may further include determining 510, via the controller, an exhalation region of the heat map based on the mouth region. The method 500 may further include determining 512, via the controller, a mask state of the monitored person based on the exhalation region and a temperature gradient classification model.

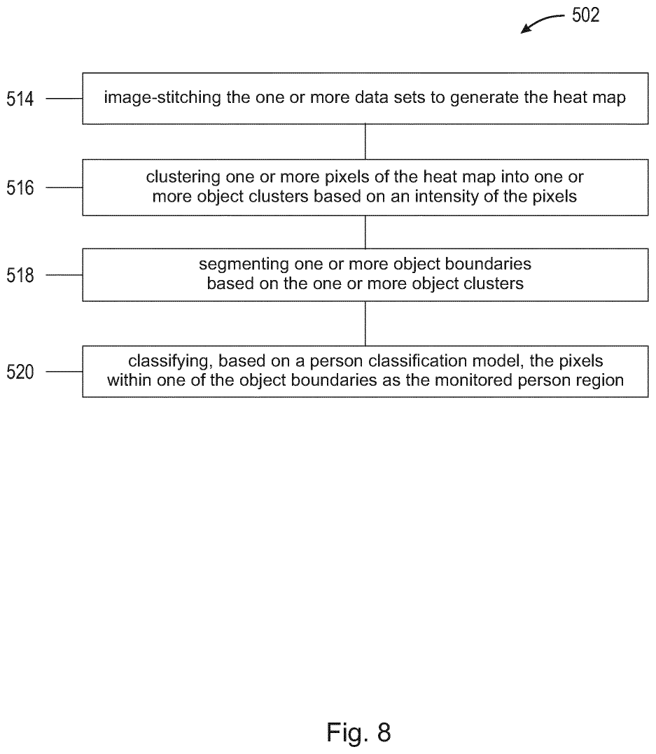

According to an example, and with reference to FIG. 8, detecting 502 the monitored person region may include image-stitching 514 the one or more data sets to generate the heat map. Detecting 502 the monitored person region may further include clustering 516 one or more pixels of the heat map into one or more object clusters based on an intensity of the pixels. Detecting 502 the monitored person region may further include segmenting 518 one or more object boundaries based on the one or more object clusters. Detecting 502 the monitored person region may further include classifying 520, based on a person classification model, the pixels within one of the object boundaries as the monitored person region.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects may be implemented using hardware, software or a combination thereof. When any aspect is implemented at least in part in software, the software code can be executed on any suitable processor or collection of processors, whether provided in a single device or computer or distributed among multiple devices/computers.

The present disclosure may be implemented as a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions may be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant may be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific examples described herein. It is, therefore, to be understood that the foregoing examples are presented by way of example only and that, within the scope of the appended claims and equivalents thereto, examples may be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A system for monitoring face mask wearing of a monitored person, comprising a controller communicatively coupled to one or more multipixel thermopile sensors, wherein the controller is configured to:
detect a monitored person region within a heat map, wherein the heat map is based on one or more data sets captured by the one or more MPTs;
locate a head region within the monitored person region;
determine a facing-direction of the head region;
locate a mouth region within the head region based on the facing-direction;
determine an exhalation region of the heat map based on the mouth region; and
determine a mask state of the monitored person based on the exhalation region and a temperature gradient classification model.

2. The system of claim 1, wherein detecting the monitored person region comprises:
image-stitching the one or more data sets to generate the heat map;
clustering one or more pixels of the heat map into one or more object clusters based on an intensity of the pixels;
segmenting one or more object boundaries based on the one or more object clusters; and
classifying, based on a person classification model, the pixels within one of the object boundaries as the monitored person region.

3. The system of claim 2, wherein the person classification model is a Light Gradient Boosting Machine.

4. The system of claim 1, wherein the head region is located by identifying a high intensity pixel cluster within the monitored person region.

5. The system of claim 1, wherein the facing-direction of the head region is determined based on a major axis of the monitored person region or a minor axis of the monitored person region.

6. The system of claim 1, wherein the exhalation region is further determined based on an audio arrival angle of one or more speech audio signals captured by one or more microphones communicatively coupled to the controller, and wherein the speech audio signals correspond to speech of the monitored person.

7. The system of claim 1, wherein the controller is further configured to transmit a warning signal based on the mask state of the monitored person.

8. The system of claim 1, wherein one or more light sources and/or one or more ionizers communicatively coupled to the controller operate in a disinfecting mode based on the mask state of the monitored person.

9. The system of claim 1, wherein the determination of the mask state of the monitored person is further based on one or more breath audio signals captured by one or more microphones and a breathing audio classification model, and wherein the breath audio signals correspond to breathing of the monitored person, and wherein the microphones are communicatively coupled to the controller.

10. The system of claim 1, wherein the temperature gradient classification model is an artificial neural network.

11. The system of claim 1, wherein the temperature gradient classification model is a support vector machine.

12. The system of claim 1, wherein the MPTs are arranged in one or more luminaires.

13. The system of claim 12, wherein the luminaires are positioned above the monitored person.

14. A method for monitoring face mask wearing of a monitored person, comprising:
detecting, via a controller communicatively coupled to one or more multipixel thermopile sensors, a monitored person region within a heat map, wherein the heat map is based on one or more data sets captured by the one or more MPTs;
locating, via the controller, a head region within the monitored person region;
determining, via the controller, a facing-direction of the head region; locating, via the controller, a mouth region within the head region based on the facing-direction;
determining, via the controller, an exhalation region of the heat map based on the mouth region; and
determining, via the controller, a mask state of the monitored person based on the exhalation region and a temperature gradient classification model.

15. The method of claim 14, wherein detecting the monitored person region comprises:
image-stitching the one or more data sets to generate the heat map;
clustering one or more pixels of the heat map into one or more object clusters based on an intensity of the pixels;
segmenting one or more object boundaries based on the one or more object clusters; and
classifying, based on a person classification model, the pixels within one of the object boundaries as the monitored person region.
